(19) United States
(12) Patent Application Publication
(10) Pub. No.: US 2006/0235539 A1
(43) Pub. Date: Oct. 19, 2006

(54) Surgical kit for hemiarthroplasty hip replacement

(76) Inventors: Gordon Blunn, Hertfordshire (GB); Allen E. Goodship, Hertfordshire (GB); Sze-Ching Fang, Taipei (TW)

Correspondence Address: Bradley N Ruben, 463 First St, Ste 5 A, Hobokern, NJ, 07030, US

(21) Appl. No.: 10/544,260

(22) PCT Filed: Feb. 3, 2004
(86) PCT No.: PCT/GB04/00406

§ 371 Date: Dec. 1, 2005

(30) Foreign Application Priority Data
Feb. 3, 2003 (GB) 0302459.3

(51) Int. Cl. A61F 2/36; A61F 2/46; A61B 17/16

(52) U.S. Cl. 623/22.12; 623/23.11; 606/81

(57) ABSTRACT

The present invention concerns apparatus and methods for hip refurbishment/replacement and in one aspect provides a kit comprising a femoral head and reamer. The present invention also provides apparatus and methods for minimally invasive surgery on a hip or similar (eg shoulder) joint. The kit is suited for performing a hip hemiarthroplasty, in which a femoral head is fitted directly into a socket reamed into the acetabular without any permanent liner or prosthetic acetabular being implanted. The reamer is used to ream out the acetabulum until cancellous bone is exposed so that it bleeds liquid containing stem cells. By selecting the size of the femoral head in accordance with the characteristics of the patient, the pressure imposed on the liquid/synovial fluid of the joint is in the range between 0.5 and 2 MPa This causes the stem cells to produce new cartilage between the bone and femoral head.

### FIELD Of THE Invention

The present invention concerns apparatus and methods for hip refurbishment/replacement and in one aspect provides a kit comprising a femoral head and reamer. The present invention also provides apparatus and methods for minimally invasive surgery on a hip or possibly even on a shoulder or other joint.

### BACKGROUND OF THE INVENTION

In conventional hip replacement surgery the surgeon replaces the femoral head and reams out the acetabulum prior to insertion of a prosthetic acetabulum. The prosthetic femoral head, stem, acetabulum and reamer are all provided as a kit by an implant supplier, such as the applicant, in accordance with a set of patient specification determined by the surgeon. The reamer is designed to ream out exactly sufficient material from the acetabulum to fit the replacement prosthetic acetabulum. This form of hip replacement is known as total hip replacement in some cases the hip is repaired by hemiarthroplasty in which the femoral head only is replaced. In such cases the natural femoral head is replaced in much the same way as with total hip replacement, however, the acetabulum is left substantially untouched with the natural cartilage still in place. Therefore the surgical kit for hemiarthroplasty does not include a reamer or acetabulum.

The prosthetic acetabulum may be said to have a male side which is closely received into the socket formed by the reamer and a female side which provides the socket into which the prosthetic femoral head directly and closely fits. The reamer is therefore sized to complement the male side of the prosthetic acetabulum.

In use the prosthetic acetabulum provides the bearing surface for the femoral head. However, being inert, the bearing surfaces of the acetabulum wear during use until the replacement hip joint needs repair. It is desirable to defer or avoid this problem completely because the repair process involves a major surgical procedure with the associated discomfort and risk to the patient.

The present invention aims, amongst other objectives, to alleviate this problem.

### SUMMARY OF THE INVENTION

According to a first aspect of the present invention there is provided a surgical kit for hip replacement comprising:

a prosthetic femoral head and a reamer,

said reamer being adapted to ream a socket into an acetabulum until the cancellous bone is exposed,

the femoral head having a size and shape complementary to the reamer such that the femoral head can be fitted closely and directly into a reamed acetabulum whereby liquid between the femoral head and the socket will be subjected to a hydrostatic pressure in the range of 0.01-5 Mpa.

The steps of the surgical procedure comprise first identifying certain characteristics of the patient which may include their weight and the dimensions of the natural femur and pelvis. Using known methods such as those developed by G. Bergman, the implant manufacturer determines the size and shape (especially radius of curvature) of femoral head required which will result in any liquid between the femoral head and a closely matching socket being subject to a pressure of between 0.01 and 5 MPa. The femoral head and matching reamer are then produced for the kit.

Detailed descriptions of the Bergman methodology, which entail radiographic assessment of the contact area of the joint and adjusting for weight and level of activity of a patient to estimate an average figure for the force across the joint are given In the following three articles, the contents of which are incorporated herein by reference : 1) Bergmann G, et al. Hip contact forces and gait patterns from routine activities. J Biomech. 2001 July;34(7):859-71. 2) Graichen F, Bergmann G, Rohimann A. Hip endoprosthesis for in vivo measurement of joint force and temperature. J Biomech. 1999 October;32(10):1113-7; and 3) Bergmann G, Graichen F, Rohlmann A. Hip joint loading during walking and running, measured in two patients. J Biomech. 1993 August;26(8):969-90. Review.

The dimensions of the reamer are chosen to closely match the dimensions of the femoral head. Generally the radius of curvature of the reamer (ie cutting envelope of the reamer) will be at most approximately 5 mm greater than that of the femoral head so that the clearance between the femoral head and acetabulum is 5 mm or less. This is broadly less than half that of a conventional total hip replacement, where an acetabular shell is inserted in the hip to serve as the bearing surface. Indeed In the preferred embodiments the radius of curvature of the reamer will be at most approximately 3 mm greater than that of the femoral head so that the clearance between the femoral head and acetabulum is 3 mm or less and may be only 1 mm to 2 mm.

In an alternative definition of the invention there is provided a surgical kit for hip replacement comprising:

a prosthetic femoral head and a reamer,

said reamer being adapted to ream a socket into an acetabulum until the cancellous bone is exposed,

the femoral head having a size and shape complementary to the reamer such that the femoral head can be fitted closely and directly into a reamed acetabulum, the size (radius of curvature) of the reamer (ie cutting envelope of the reamer) being at most approximately 5 mm greater than that of the femoral head so that the clearance between the femoral head and acetabulum is 5 mm or less.

In the surgical procedure the reamer supplied in the kit is used by the surgeon to ream out the cartilage lining the acetabulum and then the cortical bone until the underlying cancellous bone is exposed and bleeding. The liquid bleeding from the cortical bone includes stem cells. The femoral head is secured to a femoral stem socketed into the patient's natural femur in a substantially conventional way and the femoral head socketed into the reamed acetabulum.

By selecting the size of the femoral head in accordance with the characteristics of the patient, the liquid, including synovial/joint fluid, is thus subject to a hydrostatic pressure in the range of 0.01-5 MPa., preferably in the range of 0.5-2MPa and more preferably near to 2 MPa. The stem cells within the liquid are, as a result of the pressure, encouraged to form chondrocytes which grow into cartilage lying on the subchondrial bone which provides a natural active bearing surface for the femoral head, rather than relying on a rigid wholely prosthetic bearing comprising a rigid acetabular shell prosthesis as used in total joint replacement, and giving a better refined joint than a hemiarthroplasty .

In order to further promote the formation of cartilage spacers may be provided to separate the surface of the femoral head and the reamed acetabulum. The spacers may be provided on the surface of the prosthetic femoral head but preferably are mounted to the acetabular surface, and may be formed of material which is resorbable to provide a temporary bearing surface which is resorbed as the cartilage develops. Suitable example materials from which the spacers may be formed to. be resorbable are diverse and numerous. Examples include fibronectin, PLGA and polyfumarate

In a further development the kit may further include a membrane which suitably conforms to the shape of the surface of the femoral head and the reamed acetabulum. The use of a continuous/contiguous membrane as opposed to discrete spacer elements provides a more uniform and widely spread transfer of loading between the femoral head and the acetabulum. The membrane preferably also is adapted to be resorbable or is adapted to be removable from the joint once sufficient regeneration of cartilage has occurred. Preferably the membrane is formed in situ and suitably is composed of a gel/hydrogel with or without fibre reinforcement. Example fibre reinforcements may comprise polyester or fibronectin or other fibrous materials. Resorbable fibres such as fibronectin are preferred. Fibrin or fibrin glue may be used.

Other preferred compositions of the membrane include cellulose nitrate, expanded PTFE, Dacron, alginate and glycolic acid-lactic acid complex (PLGA). Polyurethane may be used on its own or with other materials and may, for example, be used in the form of a foam. Another preferred material is a collagen mesh or gel. Again, fibronectin or polyfumarate are other preferred materials usable and suited to resorbton.

The material or materials from which the femoral head surface, membrane and or spacers are formed are suitably compliant, suitably having elastohydrodynamic deformation properties, with different moduli, and suitably having different frictional characteristics to control the stresses and friction on the reamed bone surface. Particularly preferably it is the external surface of the femoral head that is compliant/pliant and as an example it may comprise a layer of polyurethane. The spacers suitably are relatively more rigid and at least more rigid than any corresponding membrane.

In those embodiments having a membrane or spacers, the membrane or spacers suitably are porous or permeable to facilitate delivery of growth factors, stem cells, chondrocytes or fibroblasts therethrough/therefrom to encourage the formation of cartilage. Example growth factors that might be used include transforming growth factor beta, insulin-like growth factor and other known cartilage growth factors. Platelet derived growth factor (PVGF) and fibroblast growth factor (FGF) may usefully be used. The growth factors, stem cells, chondrocytes or fibroblasts may suitably be bound to the spacers or membrane and released as the lafter degrade.

Where a membrane is used this may suitably be cemented in place on the femoral head and/or on the acetabular surface. The membrane may be provided with ribs or other reinforcements to counter any rucking/shear of the membrane in use. As noted above, the membrane preferably is formed in situ and suitably absorbs water for deployment. Any reinforcements may be expandable/formed in situ to facilitate deployment of the membrane using a non-invasive/minimally-invasive surgical procedure.

In a further aspect of the present invention, there is provided a non-invasive reaming procedure for joint refurbishment and which is particularly useful where neither the head of the femur nor the acetabular surface needs to be fully replaced.

In the procedure an access tunnel is first drilled through the femoral head and neck and a minimal incision is made to the (hip) joint capsule. A modular shell reamer having a separable substantially part-spherical head and shaft can be assembled in situ by introducing the shaft of the reamer through the tunnel, introducing the reamer head through the slit in the capsule and coupling the inserted end of the reamer shaft to the reamer head in situ. By use of a substantially unique double-sided reamer head, having reamer cutting teeth facing not only outwardly toward the acetabular surface but also inwardly toward the femoral head surface, both of those two surfaces may be simultaneously reamed to be thoroughly compatible with each other. The acetabular surface may be reamed by pushing the reamer via the shaft against the acetabular surface and conversely the femoral surface may be reamed by pulling the reamer shaft to force the surface of the reamer head facing the femoral surface into contact therewith. The resulting femoral head and acetabular surfaces are congruent and they define therebetween the required joint space. A membrane may be interposed between the two reamed surfaces and is suitably of the form described previously, being permanent or temporary, restorable and/or resorbable and suitably adapted to release stem cells, chondrocytes, growth factors or similar into the joint space, if required.

The access tunnel may usefully serve as a conduit for delivery of a medium for forming the membrane in situ. A biologically active gel or gelling solution such as alginate or a hydrogel suitably containing a combination of stem cells, chondrocytes, growth factors or similar may be injected into the joint space via the access tunnel. Alternatvely, the gel/medium for forming the membrane may be injected directly into the capsule rather than via the access hole.

The minimally invasive reaming method may also be employed with a single sided reaming head but to substantially less advantage.

The components of the femoral head may also include means to deliver growth factors to encourage the formation of cartilage. The spacers may deliver growth factors as they are resorbed.

### BRIEF DESCRIPTION OF THE DRAWINGS

A surgical kit for hemiarthroplasty hip replacement embodying the present invention will now be described, by way of example only, with reference to the accompanying illustrative figures, in which:

FIG. 1 illustrates a femoral head;

FIG. 2 illustrates a reamer;

FIG. 3 is similar to FIG. 1 but illustrates the apparatus in use in a hemiarthroplasty and further illustrates the provision of a membrane between the femoral head of the femoral prosthesis and the acetabulum. (FIG. 3A is a close-up of the interface between the femoral head, membrane and acetabulum);

FIG. 4A is a view similar to FIG. 3 but of a minimally-invasive surgical procedure to refurbish a hip joint when the femur is not replaced by a femoral prosthesis but where the femoral head surface and acetabular surface are each reamed in situ;and

FIGS. 4B to 4D show successive stages of the surgical procedure.

### DESCRIPTION OF THE PREFERRED EMODIMENT

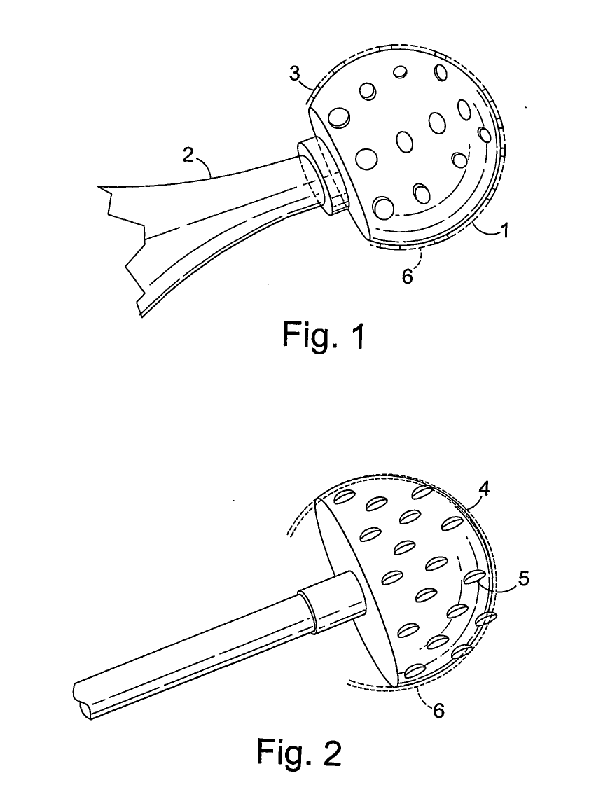

FIG. 1 illustrates a prosthetic femoral head 1 which may be integral with a femoral stem or adapted for fifting to a femoral stem as is known. A femoral shaft 2 extends from the femoral head 1. The femoral head 1 is part spherical and provided with spacers 3 located at intervals across its surface to provide a temporary bearing surface. The spacers 3 are formed from a material which is gradually resorbed and gradually dispenses growth factors.

A reamer is shown in FIG. 2 and has a part spherical head 4. Abrading elements 5 of substantially conventional design are dispersed across the spherical surface of the reamer so that in use the reamer produces a socket in an acetabulum corresponding in size and shape to the envelope indicated by the outer dotted line 6.

As can be seen in FIG. 1 the outer dotted line 6 also forms an envelope around the bearing surface provided temporarily by the spacers 3 formed on the surface of the femoral head 1. The size of the femoral head 1 is determined via reference to the characteristics such as the weight of the patient so that the pressure of liquid present between the femoral head and the socket formed In the acetabulum by the reamer is in the range between 0.01-5 MPa, and preferably close to 2 MPa, for example in the range between 0.5-2 MPa.

The femoral head fits directly into the socket produced by the reamer without any Intervening, permanent hard liner. The material from which the surface of the femoral head is made may be compliant to control the hydrostatic pressure and suitably is of polyurethane.

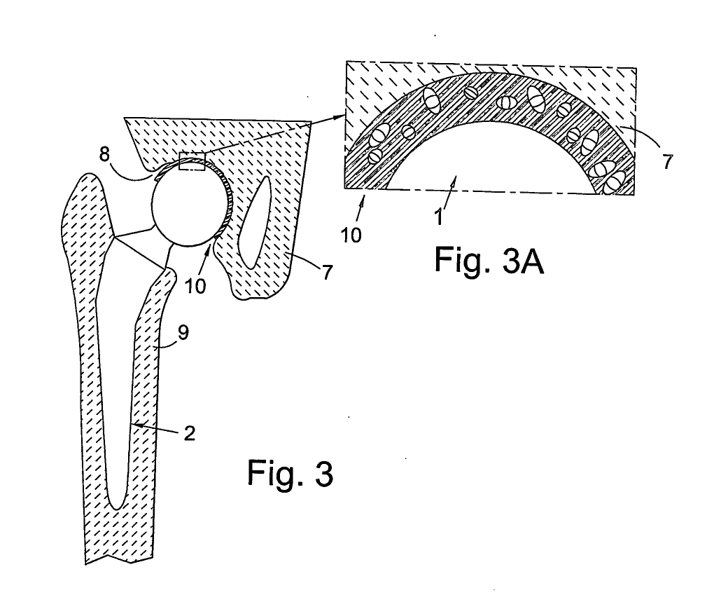

Referring to FIG. 3, the apparatus is here shown in use in a hemiarthroplasty between the acetabulum 8 of a hip 7 and the shaft of a femur 9, but is further improved by the provision of a membrane 10 between the femoral head 1 of the femoral prosthesis and the acetabulum 8. The membrane is a thin pliable membrane formed of a hydrogel and hereshown enwrapping the prosthetic femoral head 1 to transfer the loads acting across the joint more broadly and evenly than achieved by spacers alone.

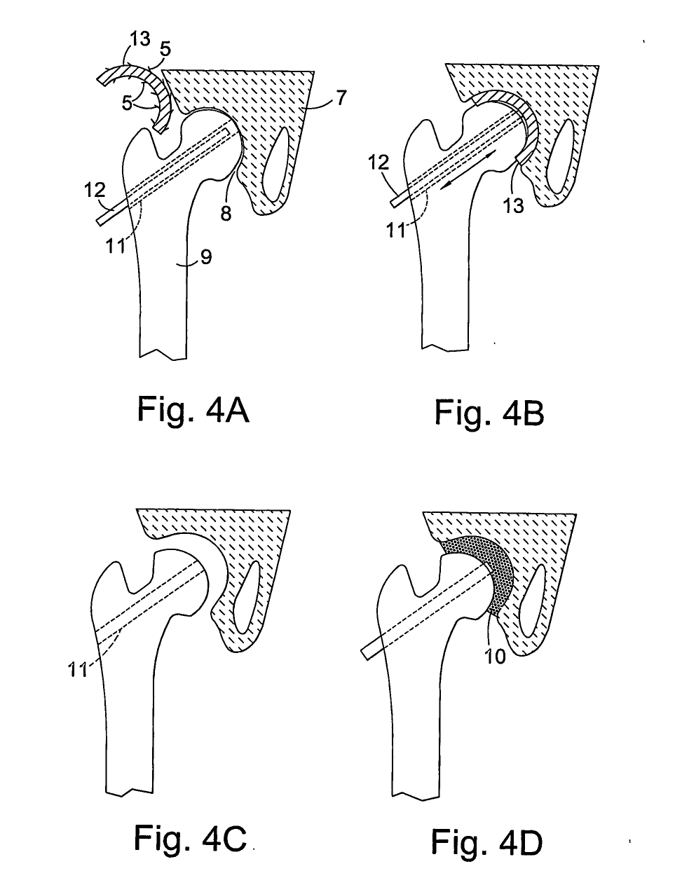

Referring to FIGS. 4A to 4D, these show the successive stages of a minimally-invasive surgical procedure to refurbish a hip joint 7,9 where the head of femur 9 is not replaced by a femoral head prosthesis and the acetabulum 8 of the hip 7 is not lined with an acetabular shell prosthesis, but where the head surface of the femur and the acetabular surface are each simply reamed in situ.

Here an access tunnel 11 is first drilled through the femur 9 head and neck and a minimal incision is made to the (hip) joint capsule. A modular shell reamer having a separable substantially part-spherical head 13 and shaft 12 can be assembled in situ by introducing the shaft 12 of the reamer through the tunnel 11, introducing the reamer head 13 through the slit in the capsule and coupling the inserted end of the reamer shaft 12 to the reamer head 13 in situ.

By use of a substantially unique double-sided reamer head 13, having reamer cutting teeth 5 facing not only outwardly toward the acetabular surface 8 but also inwardly toward the femoral head surface opposing the acetabular surface, both of those two surfaces may be simultaneously reamed to be thoroughly compatible/congruent with each other. The acetabular surface 8 may be reamed by pushing the reamer head 13 via the shaft 12 against the acetabular surface 8 and conversely the femoral surface may be reamed by pulling the reamer shaft to force the surface of the reamer head facing the femoral surface into contact therewith.

On removal of the reamer (FIG. 4C) the resulting femoral head and acetabular surfaces are congruent and they define therebetween the required joint space. A membrane 10 may be interposed between the two reamed surfaces and is suitably of the form described previously, being permanent or temporary, restorable and/or resorbable and suitably adapted to release stem cells, chondrocytes, growth factors or similar into the joint space, if required.

The access tunnel 11 may usefully serve as a conduit for delivery of a gel forming medium for forming the membrane 10 in situ. A biologically active gel or gelling solution such as alginate or a hydrogel suitably containing a combination of stem cells, chondrocytes, growth factors or similar may be injected into the joint space via the tunnel 11 or else injected directly into the capsule via the slit/incision or otherwise.

Although described hereinabove in the context of a method and apparatus for replacement/refurbishment of a hip joint, some or all of the above teachings may be applicable to other joints that have a broadly ball and socket configuration.

1. A surgical kit for hip replacement comprising:
a prosthetic femoral head and a reamer,
said reamer being adapted to ream a socket into an acetabulum until the cancellous bone is exposed,
the femoral head having a size and shape determined with reference to the weight of the patient for whom the surgical kit is intended and complementary to the reamer such that the femoral head can be fitted closely and directly into a reamed acetabulum whereby liquid between the femoral head and the socket will be subjected to a hydrostatic pressure in the range of 0.01-5 MPa.

2. A surgical kit according to claim 1, wherein the hydrostatic pressure is in the range 0.5-2 MPa.

3. A surgical kit according to claim 2, wherein the hydrostatic pressure is 2 MPa.

4. A surgical kit according to claim 1 wherein a membrane is provided which in use spaces apart the surface of the femoral head and the reamed acetabulum.

5. A surgical kit according to claim 1, wherein multiple spacers are provided which in use space apart the surface of the femoral head and the reamed acetabulum.

6. A surgical kit according to claim 4, wherein the membrane is of resorbable material.

7. A surgical kit according to claim 5, wherein the multiple spacers are of resorbable material.

8. A surgical kit according to claim 4, wherein the kit has a continuous/contiguous membrane with conforms to the shape of the surface of the femoral head and the reamed acetabulum.

9. A surgical kit as claimed in claim 4, wherein the kit has a membrane that is composed of a gel/hydrogel.

10. A surgical kit as claimed in claim 9, wherein the gel/hydrogel has fibrous materials therein for reinforcement.

11. A surgical kit as claimed in claim 4, wherein the kit has a membrane that comprises material selected from: celulose nitrate, expanded PTFE, dacron, alglnate and glycolic acid-lactic acid complex (PLGA); polyurethane; collagen mesh or gel; fibronectin; and polyfumarate.

12. A surgical kit according to claim 1, wherein the surface of the femoral head is formed from a material adapted to deform and so sustain the hydrostatic pressure.

13. A surgical kit as claimed in claim, wherein the kit comprises multiple spacers and a membrane and the membrane is less rigid than the spacers.

14. A surgical kit according to claim 4 wherein the membrane is adapted to deliver growth factors, stem cells, chondrocytes or fibroblasts to the liquid.

15. A surgical kit according to claim 5 wherein the spacers are adapted to deliver growth factors, stem cells, chondrocytes or fibroblasts to the liquid.

16. A surgical kit as claimed in claim 14, wherein said membrane is porous or permeable.

17. A surgical kit as claimed in claim 15, wherein said spacers are porous or permeable.

18. A surgical kit according to claim 1, wherein the size (radius of curvature) of the reamer (ie cutting envelope of the reamer) is at most approximately 5 mm greater than that of the femoral head so that the clearance between the femoral head and acetabulum is 5 mm or less.

19. A surgical kit comprising a reamer wherein the reamer is a modular shell reamer for joint refurbishment of a ball and socket anatomical joint such as a hip joint, having a shaft and a substantially part-spherical head separable from the shaft but capable of being securely coupled to the shaft in situ for use, the reamer head having reamer cutting teeth facing not only outwardly toward the socket surface in use but also inwardly toward the ball surface.

20. A surgical kit for hip replacement comprising:
a prosthetic femoral head and a reamer,
said reamer being adapted to ream a socket into an acetabulum until the cancellous bone is exposed,
the femoral head having a size and shape complementary to the reamer such that the femoral head can be fitted closely and directly into a reamed acetabulum, the size (radius of curvature) of the reamer (ie cutting envelope of the reamer) being at most approximately 5 mm greater than that of the femoral head so that the clearance between the femoral head and acetabulum is 5 mm or less.

21. A surgical kit according to claim 20, wherein a membrane is provided which in use spaces apart the surface of the femoral head and the reamed acetabulum.

22. A surgical kit according to claim 20, wherein multiple spacers are provided which in use space apart the surface of the femoral head and the reamed acetabulum.

23. A surgical kit according to claim 20, wherein the membrane is of resorbable material.

24. A surgical kit according to claim 20, wherein the multiple spacers are of resorbable material.

25. A surgical kit according to claim 20, wherein the kit has a continuous/contiguous membrane with conforms to the shape of the surface of the femoral head and the reamed acetabulum.

26. A surgical kit as claimed in claim 20, wherein the surface of the femoral head is formed from a material adapted to deform and so sustain the hydrostatic pressure.

27. A surgical kit as claimed in claim 20, wherein the kit comprises multiple spacers and a membrane and the membrane is less rigid than the spacers.

28. A method for hemiarthroplasty of a hip joint comprising providing a prosthetic femoral head and a reamer, and using the reamer to ream a socket into an acetabulum until the cancellous bone is exposed, the femoral head having a size and shape closely complementary to the reamer, and fitting the femoral head directly into the reamed acetabulum, the configuration being such that liquid between the femoral head and the socket will be subjected to a hydrostatic pressure in the range of 0.01-5 MP to stimulate formation of new cartilage between the bone and femoral head.

29. A minimally-invasive reaming procedure for joint refurbishment of a ball and socket anatomical joint such a hip joint, comprising forming an access tunnel through the ball part of the joint, providing a modular shell reamer having a separable substantially part-spherical head and a shaft, and introducing the shaft of the reamer through the tunnel, introducing the reamer head separately and coupling the inserted end of the reamer shaft to the reamer head in situ, the reamer head, having reamer cutting teeth facing not only outwardly toward the socket surface but also inwardly toward the ball surface, and manipulating the reamer to ream both the socket surface and the ball surface.

30. A modular shell reamer for joint refurbishment of a ball and socket anatomical joint such as a hip joint, having a shaft and a substantially part-spherical head separable from the shaft but capable of being securely coupled to the shaft in situ for use, the reamer head having reamer cutting teeth facing not only outwardly toward the socket surface in use but also inwardly toward the ball surface.

31. A method of making a surgical kit for hip replacement, the kit being as claimed in claim 1, the method comprising: determining the weight of the patient whose hip is to be replaced and estimating the contact area of the patient's hip joint required to ensure a hydrostatic pressure within the hip joint in the range of 0.01-5 Mpa and providing a prosthetic femoral head and a reamer, the reamer being adapted to ream a socket into an acetabulum until the cancellous bone is exposed, the femoral head having a size and shape complementary to the reamer and selected to ensure the required contact area such that the femoral head can be fitted closely and directly into a reamed acetabulum of the patient's hip joint whereby liquid between the femoral head and the socket will be subjected to a hydrostatic pressure in the range of 0.01-5 MPa.

32. A surgical kit for hip replacement comprising a reamer, said reamer being adapted to ream a socket into an acetabulum until the cancellous bone is exposed, the size (radius of curvature) of the reamer (ie cutting envelope of the reamer) being at most approximately 5 mm greater than that of a femoral head of a patient on whom the reamer is to be used so that the clearance between the femoral head and acetabulum is 5 mm or less.

33. A surgical kit according to claim 32, wherein a membrane is provided which in use spaces apart the surface of the femoral head and the reamed acetabulum.

34. A surgical kit according to claim 32, wherein multiple spacers are provided which in use space apart the surface of the femoral head and the reamed acetabulum.
